(19) United States
(12) Patent Application Publication
(10) Pub. No.: US 2010/0322904 A1
(43) Pub. Date: Dec. 23, 2010

(54) COMPOSITION COMPRISING BIFIDOBACTERIUM INFANTIS AND FRUCTO- AND GALACTO-OLIGOSACCHARIDES FOR THE PREVENTION OF INTESTINAL DISCOMFORT IN INFANTS

(75) Inventors: Petronella Anna Maria Johanna Scholtens, Elst (NL); Martine Sandra Alles, Apeldoorn (NL)

Correspondence Address: FOLEY AND LARDNER LLP;SUITE 500, 3000 K STREET NW, WASHINGTON, DC, 20007, US

(73) Assignee: N.V. NUTRICIA

(21) Appl. No.: 12/866,881

(22) PCT Filed: Feb. 12, 2009
(86) PCT No.: PCT/NL09/50059

§ 371 Date: Aug. 9, 2010

(30) Foreign Application Priority Data
Feb. 12, 2008 (NL) PCT/NL2008/050080

(51) Int. Cl. A61K 35/74; A61P 1/14

(52) U.S. Cl. 424/93.4

(57) ABSTRACT

Compositions comprising bifidobacteria and galactooligosaccharides and fructooligosaccharides are provided that support the introduction of solid weaning foods in the diet of infants that receive infant milk formula or breast milk.

### FIELD OF THE INVENTION

The present invention relates to compositions that support the introduction of solid weaning foods in the diet of infants that receive infant milk formula or breast milk.

### BACKGROUND OF THE INVENTION

Probiostart™ is formulated for infants and children to support a healthy digestion and immunity. One pack of probiostart contains 10 sachets, a net weight of 1.5 g/sachet and each sachet includes fructooligosaccharides and probiotic culture (Lactobacillus acidophilus Rossell-52, Bifidobacterium infantis Rosen-33 and Bifidobacterium bifidum Rosell-71).

Scholtens et al., J Pediatr Ga stroenterol Nutr, 2006, 42:553 -559 describe that addition of prebioitic oligosaccharides to solid foods induces an increase in the faecal proportion of bifodobacteria in the intestinal microbiota in the weaning period.

WO 02/060276 relates to a probiotic combination comprising different combinations of lactobacilli, propionic acid bacteria and/or bifidobacteria. The probiotics are preferably combined with a suitable prebiotic to form a synbiotic. The combination of the invention can be consumed as such or combined with a suitable foodstuff or pharmaceutical product, and it is therapeutically useful for example for stimulating the immune system and for general health improvement

### SUMMARY OF THE INVENTION

The present inventors found that the intestinal flora of infants changes significantly when introducing solid and spoonable food besides the infant milk formula or breast feeding. It was found that the percentage of Bifidobacterium infantis (B. infantis) decreased significantly while other bacteria remained stable. Additionally, the distribution of the various short chain fatty acids (SCFA's) changed after the introduction of solid foods. Particularly, the percentage of fecal acetate decreased significantly while the percentage of propionate significantly increased, see example 1.

Introducing solid foods into the infant's diet often gives rise to intestinal problems. These problems are particularly caused by changes in the intestinal environment, particularly if changes in the diet occur suddenly. The present inventors found that important characteristics are changed when solid foods are introduced. It is preferred that when solid foods are introduced in the infant's diet, e.g. besides human milk or infant milk formula, changes in the flora and SCFA patterns are reduced and smoothened so the changes occur more gradually, and that the infant does not suffer from intestinal discomfort. The present invention provides a method and a composition that reduces the changes in bifidobacteria population and SCFA levels, thereby preventing and/or reducing intestinal discomfort.

The present invention relates to the administration of B. infantis in combination with galactooligosaccharides and fructooligosaccharides in order to blunt the changes caused by the ingestion of solid weaning foods. Supplementation of B. infantis limits the reduction in the intestinal B. infantis observed when solid foods are introduced in the diet. The mixture of galactooligosaccharides and fructooligosaccharides optimally increases the acetate production, while resulting in a limited propionate production.

A main metabolic product of B. infantis is acetate. The reduction in acetate production is believed to be, at least partly, a result of the reduced occurrence of B. infantis. Hence, the co-administration B. infantis with galactooligosaccharides and fructooligosaccharides synergistically increases the acetate production, while keeping the extra proprionate production to a minimum.

The present inventors found that the mixture of galactooligosaccharides and fructooligosaccharides is more effective in stimulating acetate production, while resulting in a limited proprionate production than other prebiotics, see example 2. A limited proprionate production is desired as in infants, upon introduction of solid foods in the diet, the propionate production is already increased.

Advantageously, the combination of B. infantis, galactooligosaccharides and fructooligosaccharides is added to the solid weaning foods such as fruit purees or other foods for infants.

Preferably the present symbiotic combination is administered over a prolonged period of time, e.g. 2 or 3 months, i.e. the transitional period from infant milk formula to more solid foods. Hence, in a further aspect the present invention provides a container (e.g. a sachet) with a specific dosage of B. infantis, galactooligosaccharides and fructooligosaccharides. The present invention also provides a pack containing multiple containers with a specific dosage of B. infantis, galactooligosaccharides and fructooligosaccharides.

### DETAILED DESCRIPTION OF THE INVENTION

The weaning period is the period wherein infants gradually get accustomed to the adult solid foods. Usually infants are introduced to solid foods at the age of 4 to 6 months. Herein weaning is defined as the transition from milk feeding to solid foods. Weaning foods as used herein refers to the food composition s that are used in infants in the weaning period.

The present invention concerns the use of a composition comprising Bifidobacterium infantis and galactooligosaccharides and fructooligosaccharides for the preparation of a food product for treatment and/or prevention of intestinal discomfort in an infant due to introduction of weaning foods in the diet of said infant.

In one embodiment of the invention, the food product is prepared by combining a composition a) comprising Bifidobacterium infantis and galactooligosaccharides and fructooligosaccharides and a composition b) which is a ready-to-use food product, wherein the food product is preferably a weaning food. Preferably composition b) has a viscosity of at least 100 mPas at a shear rate of 100 s⁻¹ at 20° C. Preferably the weight ratio of composition a) to composition b) is between 0.005 and 0.1. Preferably the amount of composition a) is between 0.5 g to 10 g.

Thus the present invention also relates to a composition c) for treatment and/or prevention of intestinal discomfort in an infant due to introduction of food products in the infant's diet, wherein composition c) is prepared by combining 0.5 g to 10 g of a composition a) with a composition b) wherein:
composition a) comprises
   i. Bifidobacterium infantis; and
   ii. galactooligosaccharides and fructooligosaccharides;
composition b) is a food product having a viscosity of at least 100 mPas at a shear rate of 100 s⁻¹ at 20° C.; preferably the food product is a weaning food; and the weight ratio of composition a) to composition b) is between 0.005 and 0.1.

In a further aspect the present invention relates to a method for making a food product comprising adding a composition a) to a composition b), to obtain a composition c) wherein:
composition a) comprises
   i. Bifidobacterium infantis; and
   ii. galactooligosaccharides and fructooligosaccharides:
composition b) is a food product having a viscosity of at least 100 mPas at a shear rate of 100 s ⁻¹ at 20° C., preferably the food product is a weaning food having a viscosity of at least 100 mPas at a shear rate of 100 s ⁻¹ at 20°; and the weight ratio composition a) to composition b) is between 0.005 and 0.1.

In a further aspect the present invention relates to a packaged powder composition comprising between 0.5 and 10 g powder, including galactooli gosaccharides, fructooligosaccharides and Bifidobacterium infantis. The present invention also provides a container comprising at least 5 of such packaged compositions.

Bifidobacterium

The present composition preferably comprises Bifidobacterium infantis (B. infantis), more preferably Bifidobacterium infantis-longum. Preferably the present composition contains a bacterium which has at least 95% identity with the 16 S rRNA sequence when compared to the type strain Bifidobacterium infantis (strain LMG 8811 ^{T} species name: Bifidobacterium longum Reuter 1963 AL, ATCC 15697), more preferably at least 97% identity. A method to determine the percentage identity with the 16 S rRNA is described by Haarman and Knol in Quantitative real-time PCR assays to identify and quantify fecal Bifidobacterium species in infants receiving a prebiotic infant formula. Appl. Environ. Microbiol. 2005;71(5):2318-24. The abovementioned strain with at least 95%, preferably at least 97%, identity of the 16 S rRNA sequence is herein referred to as Bifidobacterium infantis (B. infantis).

According to a preferred embodiment, the present composition contains at least one bifidobacterium selected from the group consisting of B. longum spp. infantis M-63.

The present composition a) preferably contains 10² to 10¹³ colony forming units (cfu) B. infantis per g dry weight of the composition , preferably 10⁴ to 10¹², more preferably 10⁵ to 10¹⁰ B. infantis per g dry weight of the present composition a). Preferably, the present composition a) contains 10⁴ to 10¹², more preferably from 10⁵ to 10¹⁰ colony forming units (cfu) B. infantis per g of the total of non-digestible saccharides. The B. infantis is preferably administered at a daily dose of 10² to 10¹³, more preferably from 10⁵to 10 ¹¹ from colony forming units (cfu).

Non-digestible Oligosaccharides

The present composition a) preferably comprises galactooligosaccharides. The term “galacto-oligosaccharide” as used herein refers to a non-digestible oligosaccharide, wherein at least 30% of the saccharide units are galactose units, preferably at least 50%, more preferably at least 60%. Lactose is considered digestible. The present composition preferably comprises galacto-oligosaccharides with a DP of 2 to 100, more preferably a DP of 2 to 10. Preferably the saccharides of the galacto-oligosaccharide are β-linked. Many human milk oligosaccharide -core structures are also β-linked.

Preferably the present composition a) comprises a galacto -oligosaccharide selected from the group consisting of (trans)galacto-oligosaccharides, lacto-N-tetraose (LNT) and lacto-N-neotetraose (neo-LNT). In a particularly preferred embodiment the present composition comprises transgalacto-oligosaccharide. Transgalacto-oligosaccharides have the chemical structure of [galactose]ₙ-glucose and/or [galactose]ₙ-glucose-([galactose])ₘ wherein n and/or m is an integer from 1 up to and including 60, i.e. 2, 3, 4, 5, 6, . . . , 59, 60; preferably n is 2, 3, 4, 5, 6, 7, 8, 9 and/or 10. Preferably m is 2, 3, 4, 5, 6, 7, 8, 9 and/or 10. Preferably the present composition comprises [galactose]ₙ-glucose wherein n is an integer from 1 up to and including 60. Preferably n is 2, 3, 4, 5, 6, 7, 8, 9 and/or 10, more preferably n is 2, 3, 4, 5, 6 and/or 7. Transgalacto-oligosaccharides (TOS) are for example sold under the trademark Vivinal™ (Borculo Domo Ingredients, Netherlands) and Oligomate 55™ (Yakult). Preferably the saccharide units of the galacto-oligosaccharides are mainly β-linked.

The present composition a) preferably comprises 0.01 to 10 g of the galacto-oligosaccharides per 100 g dry weight, preferably between 0.1 and 5 g, more preferably between 0.25 and 2 g.

The present composition a) preferably contains fructooligosaccharides. The term “fructo-oligosaccharide” as used herein refers to a non-digestible polysaccharide carbohydrate comprising a chain of at least 2β-linked fructose units, with a DP of 2 to 250, preferably 7 to 100, more preferably 20 to 60. Preferably inulin is used. Inulin is available under the tradename Raftilin HP®, (Orafti). The average DP of the present fructo-oligosaccharide is preferably at least 7, more preferably at least 10, preferably below 100. The fructo-oligosaccharide used preferably has the (majority of) fructose units linked with a β(2→1) linkage. Other terms for fructooligosaccharides include inulin, fructopolysaccharide, polyfructose, fructans and oligofructose. The present composition preferably comprises fructo-oligosaccharides with a DP of 2 to 100, preferably with a DP of 7 to 60.

The present composition a) preferably comprises 0.01 to 10 g of the fructooligosaccharides pert 100 g dry weight preferably between 0.1 and 5 g, more preferably between 0.25 and 2 g.

The present composition a) thus preferably comprises at least two non-digestible oligosaccharides with different average degrees of polymerization (DP). Preferably the weight ratios are:

i. (non-digestible oligosaccharides with DP 2 to 5):(non-digestible oligosaccharides with DP 6, 7, 8, and/or 9)>1; and/or

ii. (non-digestible oligosaccharides with DP 10 to 60):(non-digestible oligosaccharides with DP 6, 7, 8, and/or 9)>1

Preferably one or both weight ratios are above 2, even more preferably above 5. These combinations provide optimal production of acetate.

For further improvement, the present non -digestible oligosaccharide preferably has a relatively high content of short chain oligosaccharides, as these strongly stimulate acetate production. Preferably at least 50 wt. %, more preferably at least 75 wt. % of the non-digestible oligosaccharides have a DP of 2 to 9 (i.e. 2, 3, 4, 5, 6, 7, 8, and/or 9).

In the present composition a) preferably at least 75 wt. % of the total galactooligo saccharides have a DP of 2-7 and at least 75 wt. % of the total fructooligosaccharides have a DP of 6-60.

The present composition a) preferably comprises 0.01 to 10 g non-digestible oligosaccharides, preferably between 0.1 and 5 g, more preferably between 0.25 and 2 g. Per 100 g dry weight???

Method

The present invention relates to a method for making a food product comprising adding a composition a) to a composition b), to obtain a composition c).

Composition a) preferably comprises Bifidobacterium infantis and (ii) galactooligosaccharides and fructooligosaccharides. Composition a) preferably has a weight below 100 grams, most preferably below 10 gram, more preferably below 5 gram. Composition a) is preferably package d in a container, preferably a sachet. Individual packaging of composition a) enables a precise dosing of the components and prevents overdosing. Composition a) is preferably in powder form. In powdered form the bacteria have an increased shelf life. The water activity of composition a) is preferably limited to increase shelf life of the bacteria. The water activity (a_{w})of composition a) is preferably below 0.5, more preferably below 0.3, most preferably below 0.2.

Composition b) preferably has a weight below 1000 g and a viscosity of at least 100 mPas at a shear rate of 100^{s-1} at 20° C. Preferably composition b) has a viscosity of at least 200 mPas, more preferably a viscosity of at least 500 mPas at a shear rate of 100^{s-1} at 20° C. Highly viscous composition s are not preferred, particularly not for infants. Hence, preferably composition b) has a viscosity below 10000 mPas, preferably below 5000 mPas at a shear rate of 100^{s-1} at 20° C. Composition b) is preferably a fruit puree, a spoonable milk based cereal composition or a meal suitable for infants in the age of 3 to 24 months. It is preferred composition b) is a ready-to-use food product, preferably composition b) is a commercially available food product.

According to the present method composition a) and composition b) are combined, to obtain a composition c), e.g. by strewing composition a) onto composition b), and optionally followed by mixing. The method preferably has a minimal influence on the taste and/or consistency of the composition b). Hence the composition a) preferably has a limited volume compared to composition b). Therefore, the weight ratio of composition a) to composition b) is below 0.1, more preferably below 0.05. The weight ratio of composition a) to composition b) is preferably above 0.005, more preferably above 0.01 and is for example about 0.025.

In a further embodiment, the present invention provides a container with multiple individually packaged compositions a). The present invention provides a container comprising at least 5 packaged powder compositions, wherein each packaged powder composition comprises 0.5 and 10 grams powder, including galactooligosaccharides, fructooligosaccharides and Bifidobacterium infantis. The container preferably contains at least 5 packaged powder compositions, preferably at most 30 packaged powder compositions.

Health Benefits

The present invention relates to a method for (i) the treatment and/or prevention of intestinal discomfort due to introduction of weaning foods in the infant's diet and/or (ii) the treatment and/or prevention of constipation and/or (iii) the treatment and/or prevention of intestinal cramps, by administering a composition c) to the infant, wherein composition c) has been prepared by combining a composition a) and a composition b). Composition a) and composition b) are described hereinabove.

The composition c) is preferably administered to an infant with an age of 3 to 24 months, more preferably to an infant with an age of 3-12 months. Preferably the composition c) is administered to the infant at least once per week, for a period of at least 5 weeks. More preferably the composition c) is administered to the infant at least three times per week, for a period of at least 2 weeks. Most preferably the composition c) is administered to the infant at least once per day, for a period of at least 2 weeks. A more frequent administration will result in a more gradual change of intestinal flora from a milk fed infant to a situation wherein the infant also receives solid foods.

### EXAMPLES

### Example 1

### Changes in the Intestinal Microbiota

Aim

It is well known that the intestinal microbiota of breast fed infants is dominated by bifidobacteria and lactobacilli. It has been suggested that the introduction of solid weaning foods may disturb the intestinal microbiota of breast fed infants. The current study is aimed at observing the changes in the intestinal microbiota, and its metabolic activity in fully breast fed infants that were introduced to regular solid weaning foods.

Methods

The study was an observational study, in which healthy, fully breast fed infants, aged 4-6 months old, were followed from the first introduction of solid foods until six weeks thereafter. Fecal samples were taken before, and 3 and 6 weeks after the introduction of solids. The fecal samples were analyzed with fluorescent in situ hybridization and quantitative real time PCR for the analysis of percentages of bifidobacteria, B. infantis, B. adolescentis, lactobacilli, E. coli, clostridia, and Bacteroides species. In addition, fecal short-chain fatty acids (S CFAs), and pH levels were analyzed. Furthermore, information on number of stools, stool characteristics and gastrointestinal complaints (possetting, diarrhea, colics, flatulence, constipation, diaper dermatitis) were recorded in daily study diaries. All data were statistically analyzed with a repeated measures ANOVA.

Results

Thirteen infants completed the study. The mean age at study entrance was 5,0 months (s.d. 0,6 months). After six weeks of consumption of solid weaning foods, a significant increase in lactobacilli from 3,3% to 6,0% (p<0,0001), and a significant increase in B. adolescentis from 0,8% to 4,1% (p<0,0001) was observed. The percentage of B. infantis decreased significantly from 35,6% to 28,5% (p<0,0001). Other bacteria remained stable throughout the study period. The distribution of the various SCFAs changed after the introduction of solid foods. The percentage of fecal acetate decreased significantly with mean percentages of 93% and 83% at baseline and after six weeks respectively (p=0.010). The percentage of propionate significantly increased from 4% at baseline to 15% (p=0.002). No significant changes in fecal pH were observed. The stool consistency significantly increased from 1.7 (SEM 0.4) (watery to soft-pudding-like) to 2.7 (SEM 0.7) (soft-formed to dry formed) within the six-week study period (p<0.001). Stool frequency did not change significantly.

TABEL 1

| | Week 0 | Week 6 | p-value |
|---|---|---|---|
| B. adolescentis | 0.8 | 4.1 | p < 0.001 |
| B. infantis | 35.6 | 28.5 | p < 0.001 |
| acetate | 93% | 83% | p = 0.010 |
| proprionate | 4% | 15% | p = 0.002 |

Conclusion

The introduction of solid weaning foods is associated with a change in the microenvironment of fully breast fed infants, as observed in a shift in the percentages of lactobacilli, B. adolescentis and B. infantis, and as observed in a change in the metabolic activity of the intestinal microbiota, with changes in the pattern of the short-chain fatty acids acetate and propionate.

### Example 2

### Effect of Oligosaccharides on Acetate/Propionate Production

Micro-organisms were obtained from fresh faeces from bottle fed babies. Fresh faecal material from babies ranging 1 to 4 month of age was pooled and put into preservative medium within 2 h. As substrate either prebiotics (TOS; TOS/inulin (HP) mixture in a 9/1 (w/w) ratio; inulin; oligofructose(OS)/inulin mixture in a 1/1 (w/w) ratio, or none (blanc) were used. The transgalactooligosaccharides (TOS) were obtained from Vivinal GOS, Borculo Domo Ingredients, Zwolle, The Netherlands). The inulin (HP) Orafti active food ingredients, Tienen, Belgium., i.e. Raftiline HP®.

The experiment was carried out using the following samples: 1) 85 mg TOS 2) 85 mg inulin 3) 85 mg TOS/inulin in a ratio of 9/1 (w/w) and 4) 85 mg OS/inulin in a ratio of 1/1 (w/w). SCFA (acetate, propionate, butyrate) were quantitated using a Varian 3800 gas chromatograph (GC) (Varian Inc., Walnut Creek, U.S.A.) equipped with a flame ionisation detector.

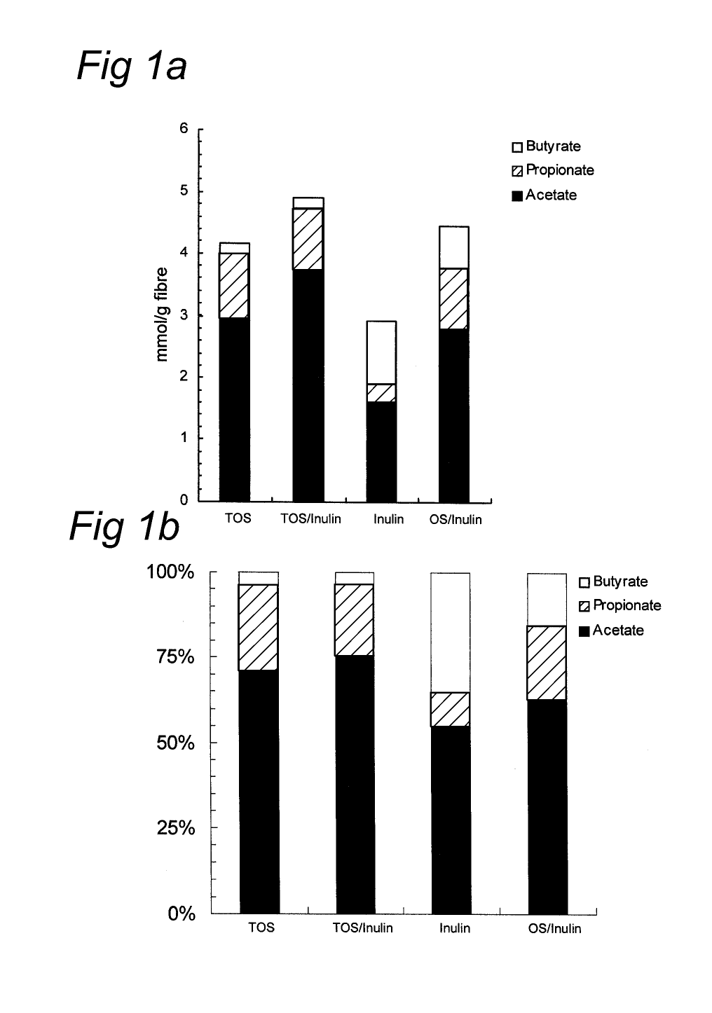

FIG. 1 depicts the absolute (FIG. 1A) and relative SCFA profile (FIG. 1B) resulting from fermenting the different oligosaccharides. FIG. 1A shows that a mixture of two different oligosaccharides (TOS/Inulin), wherein the two distinct oligosaccharides have a different chain length results in a significantly and synergistically increased amount of SCFA (particularly acetate) per gram fiber than single components. FIG. 1B shows that the addition of a combination of TOS/Inulin favored a higher proportion of the beneficial acetate (B). [FIGS 1A and 1B to be included]

### Example 3

### Composition

Packaged powder composition in sachet containing 5 g powder, including 1 g galactooligosaccharides (95 wt. % of the galactooligosaccharides have a DP of 2-7), 0.1 g fructooligosaccharides (95 wt. % of the fructooligosaccharides have a DP of 6-100)_and 10⁶ cfu B .infantis, and maltodextrin carrier.

The packaged powder composition is opened and the powder is admixed with 100 g apple puree and served to an infant of 6 months in order to prevent constipation as a result of introduction of weaning foods.

1-10. (canceled)

11. A composition comprising Bifidobacterium infantis, galactooligosaccharides and fructooligosaccharides.

12. A food product comprising (a) a ready-to-use weaning food and (b) 0.5 to 10 g of a composition comprising Bifidobacterium infantis, galactooligosaccharides and fructooligosaccharides, wherein the weight ratio of the composition to the ready-to-use weaning food is between 0.005 and 0.1.

13. The food product according to claim 11, wherein the ready-to-use weaning food has a viscosity of at least 100 mPas at a shear rate of 100 s⁻¹ at 20° C.

14. The food product according to claim 11, wherein the ready-to-use weaning food is a fruit puree, a spoonable milk based cereal composition or a meal suitable for infants in the age of 3 to 24 months.

15. The food product according to claim 11, wherein the composition is a powder.

16. The food product according to claim 11, wherein the composition has a caloric content from digestible carbohydrates, fats and proteins below 10 kcal/g.

17. The food product according to claim 16, wherein the composition has a caloric content from digestible carbohydrates, fats and proteins below 5 kcal/g.

18. The food product according to claim 17, wherein the composition has a caloric content from digestible carbohydrates, fats and proteins below 1 kcal/g.

19. A method for treatment and/or prevention of intestinal discomfort in an infant due to introduction of weaning foods in the diet of said infant, the method comprising administering to the infant a food product (a) a ready-to-use weaning food and (b) 0.5 to 10 g of a composition comprising Bifidobacterium infantis, galactooligosacchandes and fructooligosaccharides, wherein the weight ratio of the composition to the ready-to-use weaning food is between 0.005 and 0.1.

20. A package comprising 0.5 to 10 g of a powdered composition comprising Bifidobacterium infantis, galactooligosaccharides and fructooligosaccharides.

21. A container comprising at least 5 packages according to claim 20.

22. A method for making a food product comprising combining 0.5 to 10 g of a composition comprising Bifidobacterium infantis, galactooligosaccharides and fructooligosaccharides with a ready-to-use weaning food having a viscosity of at least 100 mPas at a shear rate of 100 s⁻¹ at 20° C., wherein the weight ratio of the composition to the ready-to-use weaning food is between 0.005 and 0.1.
